(19) United States
(12) Patent Application Publication
(10) Pub. No.: US 2009/0032121 A1
(43) Pub. Date: Feb. 5, 2009

(54) Check Valve

(76) Inventor: James Y. Chon, Irvine, CA (US)

Correspondence Address: ALCON, IP LEGAL, TB4-8, 6201 SOUTH FREEWAY, FORT WORTH, TX, 76134, US

(21) Appl. No.: 11/831,060

(22) Filed: Jul. 31, 2007

(51) Int. Cl. F16K 15/04; A61F 9/007

(52) U.S. Cl. 137/529; 606/107

(57) ABSTRACT

A check valve having two opposing springs to hold a ball or stopper a pre-determined distance from the valve seat so that the ball or stopper seats against the valve seat only under backflow pressure.

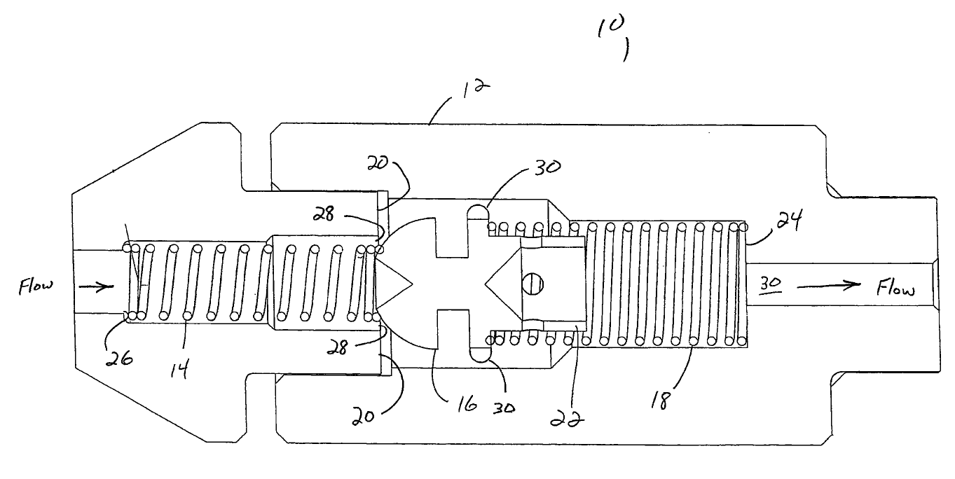

### BACKGROUND OF THE INVENTION

This invention relates generally to the field of cataract surgery and more particularly to a check valve for use in a handpiece for practicing the liquefaction technique of cataract removal.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

Recently, a new cataract removal technique has been developed that involves the injection of hot (approximately 45° C. to 105° C.) water or saline to liquefy or gellate the hard lens nucleus, thereby making it possible to aspirate the liquefied lens from the eye. Aspiration is conducted with the injection of the heated solution and the injection of a relatively cool solution, thereby quickly cooling and removing the heated solution. This technique is more fully described in U.S. Pat. No. 5,616,120 (Andrew, et al.), the entire contents of which is incorporated herein by reference. A commercially available handpiece used to practice this cataract removal technique is described in U.S. Pat. No. 6,860,868 (Sussman, et al.) and sold as the AQUALASE® handpiece by Alcon Laboratories, Inc., Fort Worth, Tex. The handpiece disclosed in this reference uses a check valve to prevent the pressurized pulses of heated fluid from being forced backwards through the handpiece toward the irrigation fluid source. The check valve has a stopper or ball that is pressed against a valve seat by a spring, so that the valve is normally closed at ambient conditions. The inventors have found that during autoclaving, mineral residue from the irrigation fluid can build up around the stopper and seat, affecting the performance of the check valve. In addition, check valves that are closed completely require some initial force to open, thereby reducing the efficiencies of the handpiece at lower operating pressures, and making the check valve very sensitive to the spring rate and load force variability.

Therefore, a need continues to exist for a check valve with increased performance and reduces sensitivity to spring rate, load force variability and mineral deposits.

### BRIEF SUMMARY OF THE INVENTION

The present invention improves upon the prior art by providing a check valve having two opposing springs to hold a ball or stopper a pre-determined distance from the valve seat so that the ball or stopper seats against the valve seat only under backflow pressure. Accordingly, one objective of the present invention is to provide a check valve having two opposing springs.

Another objective of the present invention is to provide a check valve having two opposing springs so that the check valve seals only under backflow pressure.

These and other advantages and objectives of the present invention will become apparent from the detailed description and claims that follow.

### BRIEF DESCRIPTION OF THE DRAWINGS

The FIGURE is a cross-sectional view of the check valve of the present invention.

### DETAILED DESCRIPTION OF THE INVENTION

Check valve 10 of the present invention generally includes hollow housing 12, upstream spring 14, ball or stopper 16 and downstream spring 18. Formed within housing 12 is valve seat 20. Housing 12, upstream spring 14, ball or stopper 16 and downstream spring 18 are all constructed of conventional materials well-known in the art. Spring 18 is attached to stem 22 of ball 16 and presses against surface 24 of housing 12 so as to bias ball 16 toward valve seat 20. Spring 14 contacts ball 16 near valve seat 20 and presses against surface 26 so as to bias ball 16 away from valve seat 20. At ambient conditions, the strength of spring 14 is sufficient to prevent ball 16 from sealing against seat 20 and forming small gap 28. The relative strengths of springs 14 and 18 is such that any back pressure in downstream tube 30 pushes against seal 32 on ball 16 and overcomes the biasing function of spring 14 so that ball 16 seals against seat 20. When the back pressure is removed, spring 14 once against pushes ball 16 away from seat 20, forming gap 28. Such a construction allows check valve 10 to remain open except when there is pressurized fluid in downstream tube 30, but check valve 10 closes quickly once pressurized fluid builds in downstream tube 30 as any fluid pressure acts on seal 30 to push ball 16 against seat 20 and close gap 28 before any backflow of fluid can escape out of gap 28.

This description is given for purposes of illustration and explanation. It will be apparent to those skilled in the relevant art that changes and modifications may be made to the invention described above without departing from its scope or spirit.

I claim:

1. A check valve, comprising:
a) a hollow housing;
b) a ball or stopper that reciprocates within the hollow housing;
c) a first spring biasing the ball or stopper in a first direction; and
d) a second spring biasing the ball or stopper in a second direction opposite the first direction.
